(19) United States
(12) Patent Application Publication
(10) Pub. No.: US 2018/0085342 A1
(43) Pub. Date: Mar. 29, 2018

(54) USE OF A POLYPHENOL FOR DECREASING GROWTH RETARDATION

(71) Applicant: Nutreco Nederland B.V., Boxmeer (NL)

(72) Inventors: Sandra Patricia Paredes Escobar, Lent (NL); Theo A.T.G. Van Kempen, Venray (NL)

(21) Appl. No.: 15/554,971

(22) PCT Filed: Mar. 1, 2016
(86) PCT No.: PCT/EP2016/054272

§ 371 Date: Aug. 31, 2017

(30) Foreign Application Priority Data
Mar. 2, 2015 (NL) 2014381

(51) Int. Cl. A61K 31/353; A23K 20/111; A23K 50/30; A61K 9/00

(52) U.S. Cl. CPC A61K 31/353; A61K 9/0056; A23K 50/30; A23K 20/111

(57) ABSTRACT

This disclosure pertains to the use of a polyphenol as an additive in food for decreasing a growth retardation of an animal by feeding the animal with that food. The disclosure also pertains to the use of a polyphenol as an additive in food for decreasing the weight variation in a herd of healthy animals by feeding these animals with that food.

### CROSS-REFERENCE TO RELATED APPLICATIONS

This application is a national phase entry under 35 U.S.C. § 371 of International Patent Application PCT/EP2016/054272, filed Mar. 1, 2016, designating the United States of America and published in English as International Patent Publication WO 2016/139188 A1 on Sep. 9, 2016, which claims the benefit under Article 8 of the Patent Cooperation Treaty to The Netherlands Patent Application Serial No. 2014381, filed Mar. 2, 2015.

### TECHNICAL FIELD

This application pertains to a method for decreasing a growth retardation of an animal. The application also pertains to a method of achieving a nutritionally complete animal food for use in this method, including the food itself.

### BACKGROUND

Growth retardation (including intra-uterine growth retardation) is a major concern in domestic animal production. Growth retardation appears to have a permanent stunting effect on growth during life. Often, an early small growth retardation even worsens during the lifespan of the animal, leading to a large variation of the end weight of the animals at their adult stage (in particular, at slaughter). Fetal growth restriction may not only reduce neonatal survival, but may have an effect on the efficiency of feed/forage utilization. As such, even very early small growth retardation (in absolute weight) negatively affects postnatal growth throughout the life span of the animal, may negatively affect whole body composition and meat quality, and may impair long-term health and performance.

Growth retardation is not the same as overall decreased growth performance, for example, due to ongoing infections with bacteria or parasites. Overall decreased growth performance, in principle, affects all animals in a herd, whereas growth retardation, per definition, does not affect the best growing animals in a herd. Decreased growth performance during infections can be easily treated by treating (optionally prophylactically) the underlying infection, for example, using antibiotics or anti parasiticides. In contrast, since the underlying mechanisms of growth retardation are not yet well understood, treatment thereof is not straightforward. For example, animals may show a growth retardation due to an earlier infection. Although the infection is long gone, some animals still show less growth (weight increase) than other animals. The reason for this is not clear. Knowledge of the underlying mechanisms has important implications for the complete prevention of growth retardation and is crucial for enhancing the efficiency of livestock production and animal health by such prevention. This may ultimately provide a molecular mechanism for the role of maternal nutrition on fetal programming and genomic imprinting to prevent even the earliest growth retardation and its negative effects.

Innovative interdisciplinary research in the areas of nutrition, reproductive physiology, and vascular biology will play an important role in designing the next generation of nutrient-balanced diets and developing new tools for livestock management that will enhance the efficiency of animal production and improve animal wellbeing. However, thus far, no methods are available to completely prevent growth retardation in an animal.

### BRIEF SUMMARY

Provided is a method for decreasing growth retardation in an animal, such that the negative effects thereof are diminished instead of increased. In other words, provided is a method that counteracts the normal physiological consequences of growth retardation, these normal consequences being at least an increase of the initial growth retardation.

A method has been devised in which method a polyphenol is used as an additive in food for decreasing the growth retardation of the animal by feeding the animal with that food. This disclosure can lead to a new nutritionally complete animal food comprising a polyphenol, or a mixture of different polyphenols at a dose of 0.001 (one thousandth) to 100,000 (one hundred thousand) mg polyphenol per kg of the food.

Surprisingly, it was found that by using a polyphenol as a feed additive, growth retardation, in particular, a decreased growth that is not the direct result of an ongoing infection with a micro-organism or parasite, can be decreased. It is noted that this differs from the use of polyphenols as a general growth performance enhancer, since this would lead to an increase of growth of the whole population, i.e., even in animals that do not suffer from growth retardation. The effect of decreasing growth retardation has been established by feeding the test animals a nutritionally over-complete food, to make sure that the animals can use their maximum growth capabilities without any nutritional restrictions. It was found that, under such circumstances, a polyphenol cannot provide any general growth-enhancing performance, but, on the other hand, is capable of decreasing any existing growth retardation. Apparently, the physiological processes responsible for decreasing growth retardation differ from those responsible for normal growth. Still, the result of application of this disclosure for any herd of healthy animals under circumstances of complete (not mal-) nutrition, is that, since the best growing animals under these circumstances do not show a weight increase, the weight variation within the herd decreases.

The use of a polyphenol to produce a nutritionally complete animal food comprising the polyphenol at a dose of 0.001-100,000 mg polyphenol per kg of the food, by mixing the polyphenol with an amount of carbohydrates, proteins and fats corresponding to the daily requirements hereof for the animal, can, for example, be practiced in a factory where nutritionally complete animal food is produced. Here, the carbohydrates, proteins and fat are mixed in the right proportions with the polyphenol, forming a total amount that is at least enough to feed one animal for a day. Alternatively, the method can be practiced by local food mixers by mixing a pre-manufactured pre-mix containing the polyphenol (and, optionally, other constituents such as vitamins, minerals, anti-oxidants, etc.) with components that contain the required proteins, carbohydrates and fat. In another embodiment, a polyphenol premix is used as a topical dressing for animal food at the site where the animals are actually fed. This disclosure could also be used by providing the polyphenol as a separate supplement next to the food, for example, in the form of a pill, via injection, etc.

It is noted that the use of certain diets or feed additives in order to influence growth retardation is known from the prior art. For example, Tummaruk et al. describe in in “The use of herbal medicine as an alternative antimicrobial in the feed of post-weaning piglets: A field trial,” Journal of Applied Animal Science, Vol. 2, No. 3, September-December 2009, pp. 25-31, that the use of the polyol compound colistin or the phenoxy compound berberine lead to lower retarded growth when compared with the use of the phenol compound halquinol. Lima et al. in the FASEB Journal, Vol. 28, No. 1, Supplement 1033.8 (“Effects of methylating vitamins and docosahexaenoic acid supplementation on intra-uterine growth retardation in a feed-restricted swine model”) propose to use vitamins and docosahexaenoic acid as feed additive to reduce growth retardation in malnourished piglets.

It is also noted that the anti-infective activity of polyphenols is known in the art. WO 2008/155393 and US 2008/0160000 described the use of polyphenols to treat an infection with coccids in chickens. As a result, the overall growth performance of the chickens increases. The effect on growth retardation, in particular, a decreased growth not due to an infection, is not known from these documents.

Lippens et al. in Arch. Geflügelk. 69(6) S.261-266, 2005 (“Effect of the use of coated plant extracts and organic acids as alternatives for antimicrobial growth promoters on the performance of broiler chickens”), described the use of essential oils as antimicrobials as an alternative for regular antibiotics that are commonly used as overall growth promotors, also known as AGPs (antimicrobial growth promoters). In such cases, the treatment inherently also affects the heaviest animals in the herd, showing as an increased overall growth. Moreover, as indicated hereinabove, Lippens uses essential oils in his research. None of the used compounds listed in the Lippens publication fall within the commonly accepted definition of polyphenols (see below).

### Definitions

A “polyphenol,” also known as a polyhydroxyphenol, is a compound that belongs to the structural class of natural organic chemicals, typically derived from the shikimate/phenylpropanoid and/or the polyketide pathway, featuring one or more phenolic units and deprived of nitrogen-based functions (see S. Quideau, “Why bother with polyphenols?,” a short article published online in 2011 by the Groupe Polyphenols of the Université Bordeaux; http://www.groupepolyphenols.com/the-society/why-bother-with-polyphenols/), and its synthetic functional equivalents. By definition, the group of polyphenols exclusively consist of compounds that belong to either the flavonoids (isoflavones, flavones, flavonols, anthocyanins, flavanols, flavanones), or the non-flavonoid phenolic acids, stilbenes and lignans. For this, see Spencer et al. in British Journal of Nutrition (2008), 99, 12-22 (in particular, FIG. 1) as confirmed i.a. by Scalbert et al. in Critical Reviews in Food Science and Nutrition, 45:287-306 (2005).

A “plant polyphenol” is a natural polyphenol obtained from a plant.

The term “animal” includes non-human animals such as animals belonging to families such as suidae, equidae, bovidae, ayes (including chickens, ducks, quail and turkeys), fish and crustaceans (including crabs, lobsters, crayfish and shrimps).

A “feed additive” is a component added to the regular nutrients (i.e., the food) of an animal, in particular, to its solid food or drinking water.

By use of the term “growth retardation” (also denoted as growth restriction) is meant the failure of an individual animal to develop a normal weight for his age under optimal growing conditions (i.e., without ongoing infections). Per definition, animals having a weight below average in a herd of healthy animals having a corresponding age, have a growth retardation.

A “dose of X ppm of component Y in food” means that the component Y is present at a level of X mg per kg of the food.

A “start-up phase” of an animal is the phase wherein the animal reaches, at maximum, 20% of its final weight, i.e., the regular (mean) weight of a grown up animal, in particular, at slaughter.

A “grower phase” of an animal is the phase wherein the animal weighs between 20% and 100% of its final weight.

A “nutritionally complete animal food” is a blend of various food components comprising the total daily requirements of carbohydrates, proteins and fats for this animal, i.e., the carbohydrates, proteins and fats are present in proportions and amounts such that, at a regular intake, the food meets the total daily requirements.

### BRIEF DESCRIPTION OF THE DRAWING

FIG. 1 is a graph showing that birds suffering from a growth retardation (low starting body weight) who were fed food containing the polyphenol blend responded strongly (and significantly) to the treatment (see also, Table 1).

### DETAILED DESCRIPTION

In a first embodiment, the polyphenol is, preferably, a naturally derived plant polyphenol. More than 8,000 polyphenolic compounds have been identified in various plant species. All plant phenolic compounds arise from a common intermediate, phenylalanine, or a close precursor, shikimic acid. Primarily, they occur in conjugated forms, with one or more sugar residues linked to hydroxyl groups, although direct linkages of the sugar (polysaccharide or monosaccharide) to an aromatic carbon also exist. Association with other compounds, like carboxylic and organic acids, amines, lipids and linkage with other phenol is also common.

A polyphenol according to the disclosure is selected from the group that consists of phenolic acids, flavonoids, stilbenes and lignans (having different numbers of phenol rings that they contain and/or different structural elements that bind these rings to one another). Phenolic acids are further divided into hydroxyl benzoic and hydroxyl cinnamic acids. Phenolic acids account for about a third of the polyphenolic compounds in our diet and are found in all plant material, but are particularly abundant in acidic-tasting fruits. Caffeic acid, gallic acid, ferulic acid are some common phenolic acids. Flavonoids are the most abundant polyphenols in the human diet and share a common basic structure consisting of two aromatic rings, which are bound together by three carbon atoms that form an oxygenated heterocycle. Biogenetically, one ring usually arises from a molecule of resorcinol, and the other ring is usually derived from the shikimate pathway. Stilbenes contain two phenyl moieties connected by a two-carbon methylene bridge. Most stilbenes in plants act as antifungal phytoalexins, compounds that are synthesized only in response to infection or injury. The most extensively studied stilbene is resveratrol. Lignans are diphenolic compounds that contain a 2,3-dibenzylbutane structure that is formed by the dimerization of two cinnamic acid residues.

In yet another embodiment, the polyphenol is selected from the group of flavan-3-01, flavanone, flavonolignan, stilbene and caffeic ester.

In still another embodiment, the polyphenol is used at a dose between 1 ppb and 100,000 ppm. This means that the animal food contains between 0.001 and 100,000 mg per kg of the food, in particular, any value between 0.001 and 100,000 ppm, such as 0.001, 0.002, 0.003 . . . 0.999, 1, 2, 3, 4, 5, 6, 7, 9, . . . 99993, 99994, 99995, 99996, 99997, 99998 and 99999 ppm (any intermediate integer in this range being explicitly disclosed herewith). The lower limit, in particular, when being at the level of at least 0.1 ppm, is in the range found to have a significant effect according to the disclosure, while the upper limit (i.e., up to 10% in mass) is the level found in some rich-in-polyphenol content materials such as concentrated grape juice.

In yet another embodiment, the polyphenol is used at a dose between 0.1 and 5000 ppm, in particular, between 0.1 and 400 ppm, in particular, between 1 and 300 ppm, in particular, between 1 and 250 ppm.

The polyphenol can be used during the start-up and/or grower phase of the animal. In an embodiment, the polyphenol is used for at least seven days. In particular, the polyphenol is used during the entire start-up and/or grower phases of the animal.

Instead of one single polyphenol, multiple different polyphenols can be used in the feed additive. In particular, when the polyphenol is derived from a natural plant, often a mixture of polyphenols is derived from this plant. Although each polyphenol may be purified and used as such, the mixture derived from the plant, or any other mixture of polyphenols, may also be used according to the disclosure.

The disclosure will now be explained in more detail using the following examples.

### EXAMPLES

Example 1 describes a first experiment with a polyphenol as feed additive, in this case, in chickens.

Example 2 describes the next experiment in chickens.

Example 3 describes an experiment in piglets early in the start-up phase.

Example 4 describes an experiment in piglets late in the start-up phase.

Example 5 describes an experiment with piglets in the grower phase.

### Example 1

Example 1 describes a first experiment with a polyphenol as feed additive, in this case, in chickens. A polyphenol was added to the diet (i.e., the food) of broilers for a period of 7 days starting at the age of 37 days. The animals were present in 33 pens divided over two rooms with 30 (room A) or 36 (room B) birds per pen. Twenty-two of these pens were used as a control and eleven were used for the treatment divided over both rooms. The starting weight of the animals was approximately 2700 g.

The control animals received a commercial diet, 10% over-formulated to assure that the animals could express their full genetic growth potential. The test group animals received the same diet supplemented with 175 ppm of flavan-3-ol.

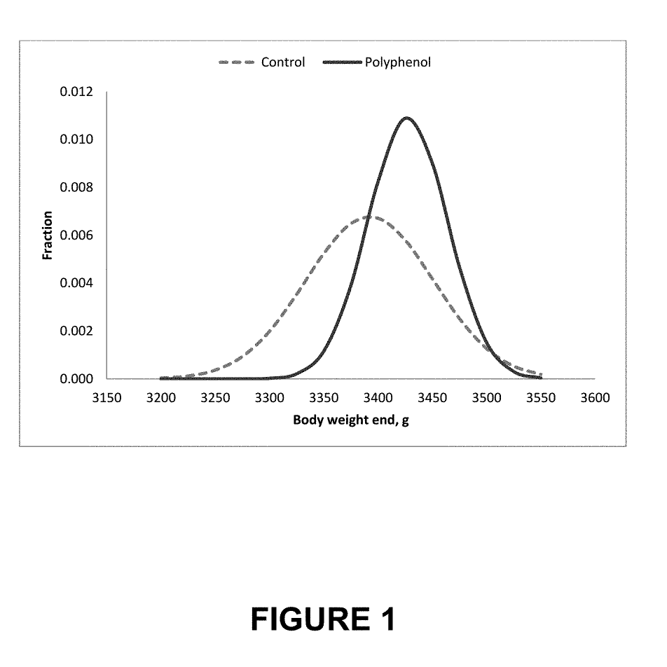

Feed intake was approximately 210 g per animal per day. The animals fed with the food containing the polyphenol blend were slightly heavier (3427 g vs. 3392 g) although this did not represent a significant increase. There was, however, a significant interaction between starting body weight and treatment: birds fed with the polyphenol blend responded strongly to the treatment when they suffered from a growth retardation (low starting body weight), while effectively no response was seen when the starting body weight was high. As a consequence, a narrower weight distribution (a smaller variation) was seen in the polyphenol-treated group. The results are indicated below in Table 1 and presented graphically in FIG. 1.

TABLE 1
Weight range covering 95% of the population, mean and standard
deviation in g

| | Mean | Stdev | Mean − 2 stdev | Mean + 2 stdev |
|---|---|---|---|---|
| Control | 3392 | 59 | 3274 | 3510 |
| Polyphenol | 3427 | 37 | 3354 | 3500 |

In this trial, the variation was notably decreased from 59 grams to 37 grams. This was mainly due to the growth-retarded animals showing a decrease in growth retardation during the treatment (the heaviest animals in the control and treatment group ending at about the same weight).

### Example 2

Example 2 describes the next experiment in chickens.

Introduction: A polyphenol was added to the diet of broilers for a period of 9 days starting at the age of 27 days. The animals were divided over 29 pens with 66 birds each. Sixteen of these pens were control pens and 13 were used for the treatment. The starting weight of the animals was approximately 1600 g. The animals were treated with the polyphenol-enriched food for 9 days. The control food was a commercial diet, over-formulated by 10% to assure that birds could express their full genetic potential. The test group received the same diet supplemented with 22, 45, 68 or 90 ppm of flavan-3-ol (equally divided over the pens).

The feed intake averaged 101 g per animal per day. Animals fed the polyphenol blend were marginally heavier (2502 g vs. 2488 g) on day 36. There was an interaction between starting body weight and treatment: birds fed with the polyphenol blend responded strongly to the treatment when the starting body weight was low, while only a modest response was seen when the starting body weight was high. As a consequence, a narrower weight distribution slightly shifted to the right was seen in the polyphenol group. See Table 2 below (data of the four polyphenol groups are combined).

TABLE 2
Weight range covering 95% of the population, mean and standard
deviation in g

| | Mean | Stdev | Mean − 2 stdev | Mean + 2 stdev |
|---|---|---|---|---|
| Control | 2488 | 34 | 2420 | 2556 |
| Polyphenol | 2502 | 26 | 2450 | 2555 |

In this trial, the variation was notably decreased from 34 grams to 26 grams. This was mainly due to the growth-retarded animals showing a decrease in growth retardation during the treatment (the heaviest animals in the control and treatment group, i.e., the “mean+2 stdev” animals, ending at about the same weight).

### Example 3

Example 3 describes an experiment with piglets early in the start-up phase. A polyphenol blend was added to the diet of nursery piglets having an age of 20 days for a period of 40 days as a partial substitute for vitamin E (kept at least at a level meeting the 2012 NRC recommendations for vitamin E in each instance). The animals were kept in 28 pens with 12 piglets each of equal age but sorted based on body weight. The starting weight was approximately 5.7 kg.

The control group received a nutritionally complete food (a so-called three-phase diet formulated to exceed the nutritional requirements) comprising 65 ppm vitamin E during the first 18 days, and 40 ppm vitamin E from days 19-40. The test group received the same nutritionally complete food supplemented with 16 ppm vitamin E and 0.7 ppm of a polyphenol blend during the first 18 days, and 11 ppm vitamin E plus 0.4 ppm of the polyphenol blend from days 19-40 (test diets are thus formulated to meet at least the 2012 NRC recommendations for vitamin E). As is commonly known, high levels of vitamin E are good for decreasing weight variation in the herd (see Van Enckevort et al. in Tijdschrift voor Diergeneeskunde, Deel 128, Februari, Aflevering 3, 2003); it was expected that lowering the amount in the control group would lead to a natural increase of weight variation. The polyphenol blend contained 38.9% flavanone, 39.9% flavonolignan, 5.2% stilbene and 16% caffeic ester.

The daily feed intake was approximately 34 g/kg body weight. The animals fed with the polyphenol blend were numerically 0.65 kg heavier on day 40. Notably, despite receiving less vitamin E, this group was substantially more homogeneous with a coefficient of variation (CV) of 7.6% vs. 11.6% for the controls. This shift appeared to be caused mainly by more rapid growth of the growth-retarded animals without a significant impact on the growth of the heavier animals, completely in line with the results seen in chickens. The results are given in Table 3.

TABLE 3
Weight range and standard deviation in kg

| | Mean | Stdev | Mean − 2 stdev | Mean + 2 stdev |
|---|---|---|---|---|
| Control | 14.6 | 1.7 | 11.2 | 18.0 |
| Polyphenol | 15.2 | 1.2 | 12.9 | 17.6 |

### Example 4

Example 4 describes an experiment in piglets late in the start-up phase. A polyphenol blend was added to the diet of nursery piglets for a period of 9 days as a substitute for vitamin E. Seven days into the supplementation, the piglets were heat-stressed for one day to try and induce (additional) growth retardation. Twenty-six piglets (half barrows, half gilts) of equal age, having a weight of approximately 12 kg at 46 days at the start of the test period were used.

The control group received a single-phase diet formulated to exceed the nutritional requirements, supplemented with 80 ppm vitamin E (common level, well above the 2012 NRC recommendations). The test group animals received the same diet supplemented with 11 ppm vitamin E (at the NRC requirements for vitamin E) plus 2 ppm of the same polyphenol blend used in Example 3.

The feed intake was approximately 3% of body weight. No substantial difference in mean weight at termination of the experiment was seen between the two treatment groups (p=0.71). The variation in weight, however, was 17.7% in the control group and 11.5% in the test group (receiving food supplemented with polyphenols).

The relationship between starting and final weight was also significantly different between the two treatment groups. This resulted in lighter test animals at the start of the trial growing relatively faster than control, while the opposite was seen for animals that entered the trial with a heavy starting weight. Data are given in Table 4.

TABLE 4
Weight range covering 95% of the population, mean and standard
deviation in kg

| | Mean | Stdev | Mean − 2 stdev | Mean + 2 stdev |
|---|---|---|---|---|
| Control | 16.1 | 2.9 | 10.4 | 21.8 |
| Polyphenol | 15.8 | 1.8 | 12.1 | 19.4 |

### Example 5

Example 5 describes an experiment with piglets in the grower phase. Flavan-3-ol was added to the diet of finisher pigs at levels of 25, 100, or 400 ppm and performance was tracked over a two-week period. For this experiment, 264 gilts and barrows were used having a starting weight of 50-55 kg, approximately at 100 days of age. The animals were treated for a period of 2 weeks.

The control animals received a single-phase diet formulated to exceed the nutritional requirements. The test animals received the same diet supplemented with 25, 100 or 400 ppm of the polyphenol. Sex interactions were not observed and removed before the final analysis. The results are indicated in Table 5.

The feed intake averaged 2.1 kg/day. It appeared that the polyphenol blend in the food increased final body weight as well as average daily gain by increasing the growth of the lightest animals in the group: pigs starting the trial at only 45 kg responded linearly to treatment; with 440 ppm polyphenols, they were 1.77 kg heavier than the controls at 14 days. In contrast, pigs starting the trial at 60 kg showed no clear treatment effect. As a consequence, the variation in body weight within the herd at day 14 was reduced.

TABLE 5
Mean body weight in kg, standard deviation and weight range
covering 95% of the population at various polyphenol levels in the food.

| | 0 ppm | 25 ppm | 100 ppm | 400 ppm |
|---|---|---|---|---|
| Mean | 64.6 | 65.5 | 65.6 | 65.8 |
| STD | 6.7 | 6.7 | 6.3 | 5.6 |
| Mean − 2 × STD | 51.3 | 52.0 | 52.9 | 54.5 |
| Mean + 2 × STD | 77.9 | 79.0 | 78.3 | 77.1 |

1. A method for decreasing growth retardation in an animal, the method comprising:
feeding the animal with food comprising an additive comprising at least one polyphenol so as to decrease growth retardation in the animal.

2. The method according to claim 1, wherein the polyphenol is a plant polyphenol.

3. The method according to claim 1, wherein the polyphenol is selected from the group consisting of flavan-3-ol, flavanone, flavonolignan, stilbene, caffeic ester, and a mixture of any thereof.

4. The method according to claim 1, wherein polyphenol is present in the food at a dose of between 1 ppb and 100,000 ppm.

5. The method according to claim 4, wherein polyphenol is present in the food at a dose of between 0.1 and 5000 ppm.

6. The method according to claim 5, wherein polyphenol is present in the food at a dose of between 0.1 and 400 ppm.

7. The method according to claim 1, wherein the food comprising the additive is fed to the animal during the start-up phase and/or grower phase of the animal.

8. The method according to claim 1, wherein the food comprising the additive is fed to the animal for at least seven days.

9. The method according to claim 7, wherein the food comprising the additive is fed to the animal for the entire start-up phase and/or grower phase of the animal.

10. The method according to claim 1, wherein the feed additive comprises multiple different polyphenols.

11. A method for decreasing the weight variation in a herd of healthy animals, the method comprising:
feeding animals in the herd with food comprising at least one polyphenol as an additive,
so as to decrease weight variation in the herd.

12. The method according to claim 1, wherein the animal belongs to the suidae.

13. The method according to claim 11, wherein the animals belong to the suidae family.

14. A method for reducing growth retardation in an animal, wherein the animal belongs to the suidae family, the method comprising:
feeding the animal with food comprising an additive comprising at least two plant polyphenols at a dose that reduces growth retardation in the animal.

15. The method according to claim 14, wherein the polyphenols are independently selected from the group consisting of flavan-3-ol, flavanone, flavonolignan, stilbene, caffeic ester, and a mixture of any thereof.

16. The method according to claim 15, wherein the dose is between 0.1 and 5000 ppm.

17. The method according to claim 16, wherein the dose is between 0.1 and 400 ppm.

18. The method according to claim 16, wherein the food comprising the additive is fed to the animal during the start-up phase and grower phase of the animal.
